SEQUENCE LISTING (2 sequences)

SEQ ID NO: 1 - DNA, 34 bp, Artificial
  Primer
ctgcagggatcctctagaggcaatgagagtgaag

SEQ ID NO: 2 - DNA, 42 bp, Artificial
  Primer
atgcatgggcccggatccctattatttttctctttgcaccac

(19) United States
(12) Patent Application Publication
(10) Pub. No.: US 2004/0162298 A1
(43) Pub. Date: Aug. 19, 2004

(54) Method of treating HIV infection by preventing interaction of CD4 and gp120

(76) Inventors: Hsu-Tso Ho, Cheshire, CT (US); Richard A. Dalterio, Chester, CT (US); Qi Guo, Newtown, PA (US); Pin-Fang Lin, Branford, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/370,820

(22) Filed: Feb. 20, 2003

Related U.S. Application Data
(60) Provisional application No. 60/359,452, filed on Feb. 23, 2002.

(51) Int. Cl. A61K 31/522

(52) U.S. Cl. 514/263.3

(57) ABSTRACT

A method of inhibiting HIV infection in a mammal by administering to said mammal in need thereof a small molecule compound having a molecular weight of less than about 1,000 dalton, wherein said compound interacts with HIV-gp120 in such a manner as to cause conformational change in said gp120 thereby preventing interaction between said gp120 and leukocyte CD4.

### REFERENCE TO RELATED APPLICATIONS

This application claims the benefit of U.S. Provisional Application Serial No. 60/359,452 filed Feb. 23, 2002.

### BACKGROUND OF THE INVENTION AND PRIOR ART

### Field of the Invention

AIDS remains a major disease that is elusive of a cure after almost two decades of intense search for an effective treatment. Currently available HIV drugs include ten reverse transcriptase and six protease inhibitors. Although drug combination regimens has results in significant decline of AIDS related death in the developed world, 78% of HIV patients with measurable viral loads carry virus that is resistant to one or more drugs (1). Furthermore, more than 20% of the newly diagnosed HIV patients are infected with resistant viruses (1). Compounds with novel anti-HIV targets are therefore urgently needed. Agents that interfere with HIV entry events represent a new class of promising inhibitors that are sought after to reinforce our arsenal in treating HIV infections (2, 3, 4, 5). HIV envelope consists of an exterior glycoprotein gp120 and a transmembrane domain gp41 (6, 7, 8). The HIV entry process involves the initial contact between the gp120 and the host cell CD4 receptor (2). Subsequent conformational changes facilitate the binding of gp120 to the coreceptor CCR5 or CXCR4 ( 9, 10, 11, 12, 13) and the insertion of the fusion peptide (14) into the host membrane, finally resulting in fusion of the virus and cell membranes (15, 16).

Agents targeting the HIV entry process can be categorized into three groups based on the mode of action: (I). GP120/CD4 binding inhibitors; (II). Co-receptor inhibitors and (III). GP41 fusion peptide inhibitors. The truncated form of CD4 (sCD4) functions as a decoy to compete with the cell associated CD4 receptor for gp120 binding; therefore the protein exhibited potent antiviral activity against HIV-1 strains tested in the laboratory. Yet, initial efforts to develop soluble CD4 as an anti-HIV agent failed in clinics due to its short serum half-life and its lack of activity against clinical HIV-1 isolates (17, 18, 19). The subsequent recombinant CD4-Ig fusion proteins (PRO542) produced by Progenic Pharmaceuticals demonstrated improved half-life in blood and achieved inhibitory activity over a broad range of HIV subtypes (20, 21). Pro 542, has entered phase II trial in an IV formulation. However an orally bioavialable anti-HIV agent targeting gp120/CD4 interaction has never been reported. Other CD4 peptide mimics (22, 23) have been shown to have affinities to gp120 in the 10 s-100 s of μM, too weak to produce significant anti-HIV activity.

Regarding co-receptor inhibitors, small cationic polypeptides such as ALX40-4C (24, 25) and T22 (26, 27) as well as cationic bicyclam (AMD3100) (28, 29) were shown to bind with coreceptor CXCR4 and inhibited HIV-1 replication. The clinical development of AMD3100 has recently been terminated due to toxicity. Significant efforts have been invested in the development of co-receptor CCR5 inhibitors. Small molecules (TAK779 (30), SCH C(31)), antibodies (PRO140 (32) ) and chemokine derivatives (33, 34) have been shown to effectively inhibit the HIV-1 entry process. Cytotoxicity may limit the clinical utility of TAK779. Proof of principle study for the Schering-Plough's CCR5 inhibitor SCH C in clinics is on going.

Finally, the viral-cell fusion inhibitors identified so far are peptidic compounds such as T20 (35) and T1249 (36) (Trimeris). T20 has shown clinical efficay in phase II trials, but it requires IV administration. ADS-J1, a cyclic inhibitor (37) has also been reported to interfere with the fusion peptide binding.

Crystal structure of a ternary complex composed of gp120 with the V1V2V3 loop-deleted the D1D2 domain CD4 and the Fab fragment of 17b (a CD4i monoclonal antibody) has been reported (38). Published PCT patent application (WO99-24065, Hendricksen et al.) (39) claimed some theoretical inhibitors that could interfere with gp120/CD4 interaction through binding with the amino acid residues located in the D1D2-CD4 binding region of gp120. The possible inhibitors claimed are purely theoretical at this time. Hendricksen et al have so far failed to produce any of the inhibitors disclosed in the PCT possessing the specified chemical characteristics and anti-HIV activity. The criteria used to specify the gp120/CD4 interaction sites were based mainly on the structural information obtained from a single gp120 conformation, generated post-CD4 and CD4i antibody binding to a loop-deleted gp120. An inhibitor that binds to gp120 and precludes a productive gp120/CD4 interaction is likely to interact with gp120 in a pre-CD4-binding conformation. For HIV to achieve entry into a host cell, this requires multiple conformation changes on gp120, CD4 and coreceptor(s). It is unlikely that the deleted regions of gp120 and CD4 disclosed by Hendricksen are important contributors to the conformational changes that are necessary to generate a productive HIV entry process. To the contrary of what Hendricksen teaches the replication efficiency of the HIV-1 strain expressing this loop-deleted gp120 has been shown not to give rise to detectably replicating viruses in cell culture (43). Therefore the entry efficiency of this envelope loop-deleted virus strain is questionable. Furthermore, the viral and cell membrane associated HIV-1 envelope is thought to be present as trimers, while the crystal structure analysis of the loop-deleted gp120 clearly showed that this gp120 is a monomer. It is clear that the amino acid residues and conformation involved in the monomer gp120/CD4 interaction as claimed in patent WO99-24065 is not automatically transferable to the trimeric gp120 interaction with CD4 receptors, without a demonstration that HIV entry into host cell CD4 is prevented thereby preventing viral replication and HIV infection.

### SUMMARY OF THE INVENTION

Applicant's invention comprises a method of inhibiting HIV infection in a mammal by administering to said mammal in need thereof a small molecule compound having a molecular weight of less than about 1000 dalton, wherein said compound interacts with HIV-gp120 in such a manner as to cause conformational change in said gp120 thereby preventing interaction between said gp120 and leukocyte CD4.

In a preferred embodiment the compound has a molecular weight of less than about 750 dalton; more preferably less than about 500 dalton.

In another preferred embodiment, the compound is administered orally to said mammal.

### BRIEF DESCRIPTION OF THE DRAWINGS

FIG. 1—Refers to the binding of [³H]BMS-853 to gp 120.

Interaction of gp120JRFL (wild type) with ³H-BMS-853. FlashPlate was coated with increase amount of gp120 and constant amount of ³H-BMS-853 (16.5 nM) in BufferC was added to each well and the binding assay was allowed to proceed as described in material and methods. Points are averages of duplicate or triplicate determinations.

FIG. 2—Refers to the binding of BMS-806 and analogs to immobilized gp120.

GP120_{JRFL} was immobilized on the surface of a CM-chip to a surface density of 12000. Inhibitors prepared in the running buffer was flown over the surface at 30 μl/min. The sensorgram was recorded and normalized against the buffer alone sensorgram, using a Biacore 3000 instrument.

FIG. 3—Refers to the binding of BMS-043 resulted in gp120 conformational change.

CD spectra was collected on a Jasco J-720 spectropolarimeter at ambient temperature. Protein concentration in the sample were kept a ˜1 μM, with the exception of sCD4 (1.8 μM) in 20 mM Tris/150 mM NaCl, pH 8.0. Concentration of compound used in the experiments were as indicated.

FIG. 4—Refers to soluble CD4 competed with [³H]BMS-853 for gp120 binding.

Competition binding of ³H-BMS-853 binding to gp120 by soluble CD4.(sCD4) ³H-BMS-853 bound to gp120 in the presence of varying amount of sCD4 was determined as described under material and methods. Points shown were obtained in a signal experiment, performed in duplicate. Data were analyzed with Prism computer program. (GraphPad Software, Inc).

### DETAILED DESCRIPTION OF THE INVENTION

The small molecule compounds with molecular weight less than about 1000 dalton interact with gp120 causing conformational change thereto and effectively block the HIV entry process by interfering with the gp120 and CD4 interaction. Typical compounds useful herein are disclosed in U.S. Pat. No. 6,476,034 issued Nov. 5, 2002 (corresponding to PCT WO 01/62255 published Aug. 30, 2001) and U.S. Pat. No. 6,469,006 issued Oct. 22, 2002 (corresponding to PCT WO 00/76521 published Dec. 21, 2000). Radioactive inhibitor, [³H]BMS-853 has been shown to bind effectively to the full-length recombinant gp¹²⁰_{JRFL}, yet bound significantly less effective to the respective V1V2V3 loop-deleted gp¹²⁰_{JRFL}. (This closely resembles the Hendricksen truncated gp120 structure). The binding of an inhibitor to the full length gp120 resulted in conformational changes in gp120 as detected by circular dichroism spectropolarimetry analysis. Due to the novel anti-HIV target and mode of action, these inhibitors are expected to be active against viral strains resistant to the current drugs. The availability of a small molecular inhibitor should also allow the oral dosing formulation, which is critical for patient compliance and enhance quality of patient life.

To summarize, the novel method for preventing HIV infection involves using small molecular weight compounds that:

Inhibits CD4 binding to full length gp120

Prevents HIV entry into host cells and syncitia formation

Binds directly to gp120

Mediates gp120 conformational change that is likely to interfere with the productive CD4 binding and subsequent events necessary for HIV entry/fusion.

References Cited

1. Richman et al. 41^{st} ICAAC, December 2001.

2. Doms, R. W. and Moore, J. P., 2000. HIV-1 membrane fusion. Targets of opportunity. J. Cell. Biol. 151 (2), F9-F14.

3. D'Souza, M. P., Cairns, J. S. and Plaeger, S. F., 2000. Current evidence and future directions for targeting HIV entry: therapeutic and prophylactic strategies. JAMA 284 (2), pp. 215-222.

4. Moore, J. P. and Stevenson, M., 2000. New targets for inhibitors of HIV-1 replication. Nat. Rev. Mol. Cell. Biol. 1, pp. 40-49.

5. Wyatt, R. and Sodroski, J., 1998. The HIV-1 envelope glycoproteins: fusogens, antigens, and immunogens. Science 280 (5371), pp. 1884-1888.

6. Earl, P. L., Doms, R. W. and Moss, B., 1990. Oligomeric structure of the human immunodeficiency virus type 1 envelope glycoprotein. Proc. Natl. Acad. Sci. USA 87 (2), pp. 648-652.

7. Kowalski, M., Potz, J., Basiripour, L., Dorfman, T., Goh, W. C., Terwillinger, E., Dayton, A., Rosen, C., Haseltine, W. and Sodroski, J., 1987. Functional regions of the envelope glycoprotein of human immunodeficiency virus type 1. Science 237, pp. 1351-1355.

8. Wyatt, R., Desjardin, E., Olshevsky, U., Nixon, C., Binley, J., Olshevsky, V. and Sodroski, J., 1997. Analysis of the interaction of the human immunodeficiency virus type 1 gp120 envelope glycoprotein with the gp41 transmembrane glycoprotein. J. Virol. 71 (12), pp. 9722-9731.

9. Berger, E. A., Murphy, P. M. and Farber, J. M., 1999. Chemokine receptors as HIV-1 coreceptors: roles in viral entry, tropism, and disease. Annu. Rev. Immunol. 17,pp. 657-700.

10. Chabot, D. J., Zhang, P. F., Quinnan, G. V. and Broder, C. C., 1999. Mutagenesis of CXCR4 identifies important domains for human immunodeficiency virus type 1 X4 isolate envelope-mediated membrane fusion and virus entry and reveals cryptic coreceptor activity for R5 isolates. J. Virol. 73 (8), pp. 6598-6609.

11. Doms, R. W., 2000. Beyond receptor expression: the influence of receptor conformation, density, and affinity in HIV-1 infection. Virology 276 (2), pp. 229-237.

12. Lee, B., Sharron, M., Blanpain, C., Doranz, B. J., Vakili, J., Setoh, P., Berg, E., Liu, G., Guy, H. R., Durell, S. R., Parmentier, M., Chang, C. N., Price, K., Tsang, M. and Doms, R. W., 1999. Epitope mapping of CCR5 reveals multiple conformational states and distinct but overlapping structures involved in chemokine and coreceptor function. J. Biol. Chem. 274 (14), pp. 9617-9626.

13. Moore, J. P., Trkola, A. and Dragic, T., 1997. Co-receptors for HIV-1 entry. Curr. Opin. Immunol. 9 (4), pp. 551-562.

14. Furuta, R. A., Wild, C. T., Weng, Y. and Weiss, C. D., 1998. Capture of an early fusion-active conformation of HIV-1 gp41. Nat. Struct. Biol. 5 (4), pp. 276-279, (erratum: 5 (7) (1998) 612).

15. Gallaher, W. R., 1987. Detection of a fusion peptide sequence in the transmembrane protein of human immunodeficiency virus. Cell 50, pp. 327-328.

16. Melikyan, G. B., Markosyan, R. M., Hemmati, H., Delmedico, M. K., Lambert, D. M. and Cohen, F. S., 2000. Evidence that the transition of HIV-1 gp41 into a six-helix bundle, not the bundle configuration, induces membrane fusion. J. Cell. Biol. 151 (2), pp. 413-424.

17. Brighty, D. W., Rosenberg, M., Chen, I. S. and Ivey-Hoyle, M., 1991. Envelope proteins from clinical isolates of human immunodeficiency virus type 1 that are refractory to neutralization by soluble CD4 possess high affinity for the CD4 receptor. Proc. Natl. Acad. Sci. USA 88 (17), pp. 7802-7805.

18. Daar, E. S., Li, X. L., Moudgil, T. and Ho, D. D., 1990. High concentrations of recombinant soluble CD4 are required to neutralize primary human immunodeficiency virus type 1 isolates. Proc. Natl. Acad. Sci. USA 87 (17), pp. 6574-6578.

19. Schacker, T., Collier, A. C., Coombs, R., Unadkat, J. D., Fox, I.., Alam, J., Wang, J. P., Eggert, E. and Corey, L., 1995. Phase I study of high-dose, intravenous rsCD4 in subjects with advanced HIV-1 infection. J. Acquir. Immune Defic. Syndr. Hum. Retrovirol. 9 (2), pp. 145-152.

20. Shearer, W. T., Israel, R. J., Starr, S., Fletcher, C. V., Wara, D., Rathore, M., Church, J., DeVille, J., Fenton, T., Graham, B., Samson, P., Staprans, S., McNamara, J., Moye, J., Maddon, P. J. and Olson, W. C., 2000. Recombinant CD4-IgG2 in human immunodeficiency virus type 1-infected children: phase ½ study (in process citation). J. Infect. Dis. 182 (6), pp. 1774-1779.

21. Jacobson, J. M., Lowy, I., Fletcher, C. V., ONeill, T. J., Tran, D. N., Ketas, T. J., Trkola, A., Klotman, M. E., Maddon, P. J., Olson, W. C. and Israel, R. J., 2000. Single-dose safety, pharmacology, and antiviral activity of the human immunodeficiency virus (HIV) type 1 entry inhibitor PRO 542 in HIV-infected adults. J. Infect. Dis. 182 (1), pp. 326-329.

22. Dowd, C. S., Zhang, W., Li, C., and Chaiken, I. M., 2001. From receptor recognition mechanisms to bioinspired minetic antagonists in HIV-1/cell docking. J. Chromatograph. B, 753, pp.327-335

23. Li, C., Dowd, C. S.,, Zhang, W., and Chaiken, I. M., 2001. Phage randomization in a charybdotoxin scaffold leads to CD4-mimetic recognition motifs that bind HIV-1 envelope through non-aromatic sequences. J. Peptode. Res. 57, pp.506-518.

24. O'Brien, W. A., Sumner-Smith, M., Mao, S. H., Sadeghi, S., Zhao, J. Q. and Chen, I. S., 1996. Anti-human immunodeficiency virus type 1 activity of an oligocationic compound mediated via gp120 V3 interactions. J. Virol. 70 (5), pp. 2825-2831.

25. Doranz, B. J., Grovit-Ferbas, K., Sharron, M. P., Mao, S. H., Goetz, M. B., Daar, E. S., Doms, R. W. and O'Brien, W. A., 1997. A small-molecule inhibitor directed against the chemokine receptor CXCR4 prevents its use as an HIV-1 coreceptor. J. Exp. Med. 186 (8), pp. 1395-1400.

26. Tamamura, H., Imai, M., Ishihara, T., Masuda, M., Funakoshi, H., Oyake, H., Murakami, T., Arakaki, R., Nakashima, H., Otaka, A., Ibuka, T., Waki, M., Matsumoto, A., Yamamoto, N. and Fujii, N., 1998. Pharmacophore identification of a chemokine receptor (CXCR4) antagonist, T22 ([Tyr(5,12),Lys7]-polyphemusin II), which specifically blocks T cell-line-tropic HIV-1 infection. Bioorg. Med. Chem. 6 (7), pp. 1033-1041.

27. Murakami, T., Zhang, T. Y., Koyanagi, Y., Tanaka, Y., Kim, J., Suzuki, Y., Minoguchi, S., Tamamura, H., Waki, M., Matsumoto, A., Fujii, N., Shida, H., Hoxie, J. A., Peiper, S. C. and Yamamoto, N., 1999. Inhibitory mechanism of the CXCR4 antagonist T22 against human immunodeficiency virus type 1 infection. J. Virol. 7 (9), pp.7489-7496

28. Donzella, G. A., Schols, D., Lin, S. W., Este, J. A., Nagashima, K. A., Maddon, P. J., Allaway, G. P., Sakmar, T. P., Henson, G., De Clercq, E. and Moore, J. P., 1998. AMD3100, a small molecule inhibitor of HIV-1 entry via the CXCR4 co-receptor. Nat. Med. 4 (1), pp. 72-77.

29. Schols, D., Este, J. A., Henson, G. and De Clercq, E., 1997. Bicyclams, a class of potent anti-HIV agents, are targeted at the HIV coreceptor fusin/CXCR-4.Antiviral Res. 35 (3), pp. 147-156.29.

30. Baba, M., Nishimura, O., Kanzaki, N., Okamoto, M., Sawada, H., Iizawa, Y., Shiraishi, M., Aramaki, Y., Okonogi, K., Ogawa, Y., Meguro, K. and Fujino, M., 1999. A small-molecule, nonpeptide CCR5 antagonist with highly potent and selective anti-HIV-1 activity. Proc. Natl. Acad. Sci. USA 96 (10), pp. 5698-5703.30.

31. Strizki J M, Xu S, Wagner N E, Wojcik L, Liu J, Hou Y, Endres M, Palani A, Shapiro S, Clader J W, Greenlee W J, Tagat J R, McCombie S, Cox K, Fawzi A B, Chou C C, Pugliese-Sivo C, Davies L, Moreno M E, Ho D D, Trkola A, Stoddart C A, Moore J P, Reyes G R, Baroudy B M. 2002. SCH-C (SCH 351125), an orally bioavailable, small molecule antagonist of the chemokine receptor CCR5, is a potent inhibitor of HIV-1 infection in vitro and in vivo. Proc Natl Acad Sci U S A. 98 (22), pp.12718-23.

32. Olson, W. C., Rabut, G. E., Nagashima, K. A., Tran, D. N., Anselma, D. J., Monard, S. P., Segal, J. P., Thompson, D. A., Kajumo, F., Guo, Y., Moore, J. P., Maddon, P. J. and Dragic, T., 1999. Differential inhibition of human immunodeficiency virus type 1 fusion, gp120 binding, and CC-chemokine activity by monoclonal antibodies to CCR5. J. Virol. 73 (5), pp. 4145-4155.

33. Mosier, D. E., Picchio, G. R., Gulizia, R. J., Sabbe, R., Poignard, P., Picard, L., Offord, R. E., Thompson, D. A. and Wilken, J., 1999. Highly potent RANTES analogues either prevent CCR5-using human immunodeficiency virus type 1 infection in vivo or rapidly select for CXCR4-using variants. J. Virol. 73 (5), pp. 3544-3550.

34. Sabbe, R., Picchio, G. R., Pastore, C., Chaloin, O., Hartley, O., Offord, R. and Mosier, D. E., 2001. Donor- and ligand-dependent differences in C—C chemokine receptor 5 reexpression. J. Virol. 75 (2), pp. 661-671.

35. Kilby, J. M., Hopkins, S., Venetta, T. M., DiMassimo, B., Cloud, G. A., Lee, J. Y., Alldredge, L., Hunter, E., Lambert, D., Bolognesi, D., Matthews, T., Johnson, M. R., Nowak, M. A., Shaw, G. M. and Saag, M. S., 1998. Potent suppression of HIV-1 replication in humans by T-20, a peptide inhibitor of gp41-mediated virus entry (see comments). Nat. Med. 4 (11), pp. 1302-1307.

36. LaBranche, C. C., Galasso, G., Moore, J. P., Bolognesi, D. P., Hirsch, M. S. and Hammer, S. M. 2001. Antiviral Research, 50, pp.95-115.

37. Debnath, A. K., Radigan, L. and Jiang, S., 1999. Structure-based identification of small molecule antiviral compounds targeted to the gp41 core structure of the human immunodeficiency virus type 1. J. Med. Chem. 42 (17), pp. 3203-3209.

38. Kwong, P. D., Wyatt, R., Robinson, J., Sweet, R. W., Sodroski, J. and Hendrickson, W. A., 1998. Structure of an HIV gp120 envelope glycoprotein in complex with the CD4 receptor and a neutralizing human antibody (see comments). Nature 393 (6686), pp. 648-659.

39. Kwong, P. D., Hendricksen et al 1998. Compounds inhibiting CD4-gp120 interaction and uses thereof. PCT WO99/24065. 1998.

40. Paul-N L; Marsh-M; McKeating-J A; Schulz-T F; Liljestrom-P; Garoff-H; Weiss-R A AIDS-Res-Hum-Retroviruses. Expression of HIV-1 envelope glycoproteins by Semliki Forest virus vectors. 1993, October; 9(10): 963-970.

41. Liljestrom P and Garoff H: A new generation of animal cell expression vectors based on the Semliki Forest virus replicon. Biotechnology 1991; 9:1356-1361.

42. Lin et al. J. of Inf. Diseases 1994: 170:1157-1164

43. Cao, J., Sullivan, N., Desjardin E., Parolin, C., Robinson, J., Wyatt, R., Sodroski, J. Replication and neutralization of human immunodeficiency virus type 1 lacking the V1 and V2 variable loops of the gp120 envelope glycoprotein. J Virol. 1997 December, 71(12): 9808-12.

TABLE 1
Inhibition of gp120_{JRFL}/CD4 Binding

| | | Anti-HIV single | gp120_{JRFL}/CD4 |
|---|---|---|---|
| | | cycle infection | Binding IC50 |
| Compound | Structure | EC50 (nM) | (μM) |
| BMS-216 | 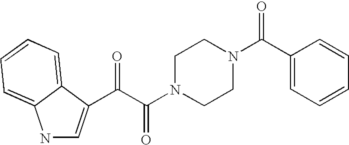 | 200 | 1.8 ± 0.3 |
| BMS-853 | 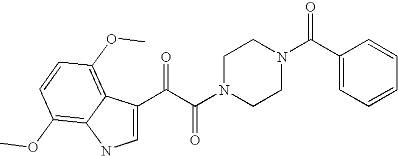 | 0.1 | 0.024 ± 0.009 |
| BMS-806 | 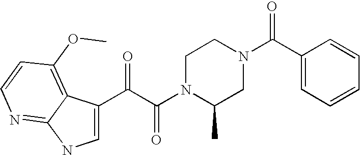 | 0.6 | 0.28 ± 0.04 |

The effect of BMS-806 on the gp120/CD4 binding. The gp120_{JRFL} in supernatant was captured onto anti-gp120 antibody (D7324) coated plate. The compound and soluble CD4 were added to the plate simultaneously and CD4 bound to gp120 was detected by ELISA using anti-CD4 antibody OKT4 as a primary antibody and the secondary antibody goat-anti-mouse-peroxidase conjugate. Value are the means±S.E.M which represent multiple separate experiments.

IC₅₀ values show concentration of compound needed to inhibit 50% of binding of gp120 and CD4.

EC₅₀ values show concentration of compound needed to inhibit 50% HIV viral replication.

Table 1 shows the correlation between more effective inhibition of binding between gp120 and CD4 provides more effective inhibition of HIV infection. Thus, compound BMS-853 is most effective.

TABLE 2
Activity of BMS-806 Against Laboratory
Adapted Strains of HIV-1

| Virus | Coreceptor | Host | EC₅₀ (nM) |
|---|---|---|---|
| JRFL | CCR5 | PM1 | 1.47 |
| LAV | CXCR4 | MT-2 | 1.98 |
| NL4-3 | CXCR4 | MT-2 | 2.94 |
| Bru | CXCR4 | MT-2 | 3.26 |
| SF-162 | CCR5 | PM1 | 3.46 |
| Bal | CCR5 | Macrophage | 8.4 |
| Bal | CCR5 | PM1 | 15.5 |
| A0-18 | CXCR4 | MT-2 | 23.6 |
| SF-2 | CXCR4 | MT-2 | 26.5 |
| IIIB | CXCR4 | MT-2 | 39.4 |
| MN | CXCR4 | MT-2 | 844 |
| RF | CXCR4 | MT-2 | 6034 |

Materials and Methods for Determination of Results Shown in Tables and Figures:

Compounds

BMS-216, BMS-806, BMS-853, BMS-033, BMS-043, BMS-003 and BMS-038 were synthesized by Bristol Myers Squibb. [³H]BMS-853 was prepared at the NTLF by the tritiation of the corresponding dibromoderivative with T2 over Pd/C in THF with triethylamine. The final compound was purified by preparative HPLC.

Cells, Viruses and Protein

Baby Hamster Kidney (BHK-21) and 293 (CRL-1573) cells were purchased from the American Type Culture Collection (ATCC) and maintained according to the suggested protocol. MT-2 and PMI cells as well as all laboratory HIV isolates were obtained from the NIH AIDS Repository. D7324 (Cliniqa, Fallbrook, Calif.) is an affinity purified sheep polyclonal antibody raised against a 15 amino acid peptide from the conserved carboxy terminus of HIV-1(LAV-1) gp120. D7324 binds to gp120 from a wide range of HIV-1 isolates. OKT4 is a murine monoclonal antibody against CD4, and was purified from an OKT4 producing hybridoma cell line (ATCC.CRL-8002). Soluble CD4, anti-gp120-HRP and goat anti mouse Ig-HRP conjugates were obtained from S ImmunoDiagnostics, Inc. (Woburn, Mass.). mV-1 gp120IIIB purified protein was purchased from Advanced Biotechnologies Inc (ABI).

Cloning and Expression of HIV-1 gp120

PCR Cloning of HIV-1 gp120 into Semliki Forest Virus Vector.

The envelope-encoding genes from the JRFL strain were PCR amplified from proviral DNA (pNL JRFL, WB) with primer KGJRFLENV5BX (CTGCAGGGATCCTCTAGAGGCAATGAGAGTGAAG) and primer KGjrflgp120.3

(ATGCATGGGCCCGGATCCCTATTATTTTTCTCTTTGCACCAC), isolated as a 1520 bp BamHI fragment and cloned into BamHI site of pSFV-SNA plasmid vector(40). pSFV-SNA is a derivative of pSFV-1(41), made by inserting a SpeI-Apal-NruI-SpeI linker into a SpeI site of pSFV-1 plasmid.

RNA Transcription

pSFV1-gp120 DNA was linearized by NruI restriction enzyme digestion and purified by a Qiagen kit. Transcription reactions were carried out using an Ambion kit following the manufacture's instructions.

Transfection and Protein Expression in BHK Cells

Electroporation was performed as originally described (Instruction manual of SFV gene Expression System) with minor modification. Briefly, cells cultured in Glasgow Minimum Essential Medium (G-MEM) containing 2 mM glutamine, 10 mM HEPES and 5% FBS was washed with versene (Gibco) and detached by 0.25% trypsin, 1 mM EDTA. After washing with G-MEM, the cells were resuspended in PBS buffer at 1.25×10⁷ cells/mi. Eight hundred microliter of cells and RNA from the transcription reaction were added into a 0.2-cm electroporation cuvette (Bio-Rad, Richmond, Calif.). Electroporation was carried out at room temperature, using a Bio-Rad Gene Pulses with two pulses at 960 μF/260V sitting. Cells were then diluted into 24 ml of complete G-MEM medium and plate in 75 cm² tissue culture Flask and incubate at 37° C. Supernatants were collected after 24 hours, 48 hours and 72 hours and quantitated with a gp120 capturing ELISA.

Alternatively, transfection was performed using a cationic lipid, DMRIE-C (Life Technologies, Inc.) which was optimized for RNA transfection. BHK-21 cells were seeded at 2×10⁵ cells per well of a 6-well plate in DMEM+5% FBS and incubated at 37° C. overnight. The next day, the monolayer was washed with OPTI-MEM (serum free, Life Technologies, Inc.). Meanwhile, RNA-lipid complexes were prepared by combining the DMRIE-C reagent with the transcribed gp120 RNA and then added directly to the cells. After incubation for 4 hours at 37° C., the complexes were washed away and the cells were placed under fresh OPTI-MEM+1% FBS. Supernatants were harvested at 24, 48, and 72 hours post transfection. Fresh OPTI-MEM+1% FBS was replenished after each supernatant removal.

Expression of gp¹²⁰_{JRFL} and Purification.

Cells at 50-60% confluence were transiently transfected with pJRFL syn gp120 (NIH AIDS Research and Reference Reagent Program) containing the HIV-1 envelope gene using LipfectAMINE PLUS reagent as described by the manufacturer (Life Technologies, Gaithersburg, Md.). Culture supernatant/medium was collected after 48 and 96 hours at 37° C. under 5% CO₂ and stored at −70° C. till protein purification was carried out.

Supernatant/medium containing gp120_{JRFL} were centrifuged at 3000 rpm for 10 min and the cell debris free supernatant was concentrated down to ˜50 ml using an Amicon ultrafiltration chamber lined with a YM30 membrane (Millipore, Beford, Mass.) under house air pressure at RT. The concentrate was dialyzed twice against dialysis buffer (20 mM Tris-HCl, pH 8.0, 0.15 M NaCl, 0.5 mM MgCl₂, 0.5 mM CaCl₂). The gp120 protein was absorbed onto 10 ml of lectin Sepharose 4B (Pharmacia, Peapack, N.J.), pre-equilibrated with 10-volume of buffer A (20 mM Tris-HCl, pH 8.0, 0.5 mM MgCl₂, 0.5 mM CaCl₂) at 4° C. at 1 ml/min. The column was first washed with the buffer A containing 1 M NaCl. and gp120 was eluted with the elution buffer containing 20 mM Tris-HCl, pH 8.0, 0.5 M α-methyl mannoside, 0.5 M a-methyl glucoside and 0.1 mM EDTA. The eluent collected was twice dialyzed against 25 mM Tris-HCl, pH 7.6 and then concentrated to 10 ml using a Amicon ultrafiltration chamber lined a YM 30 filter membrane. Protein was further purified through a ready-packed HiTrap Q column (Pharmacia, Peapack, N.J.) on a Pharmacia LKB FPLC. After loading protein onto the column and a wash with Buffer A, gp120 was eluted with a 0-400 mM NaCl gradient. Fractions containing gp120 were pooled and concentrated using a Centricon YM30 (Millipore) filter at 4° C. Protein solution were stored in small aliquots at 4° C.

Gp120 Capture ELISA

The gp120 capturing ELISA methods was derived from Moore et al (42). Briefly, the 96 well assay plates (Immulon 4, Dynatech Technologies Inc. Chantilly, Va.) were coated overnight at 4° C. with 100 μl per well of 10 ug/ml D7324 solution in 100 mM bicarbonate buffer (pH9.6). The antibody-coated plate was washed twice with TBS (20 mM Tris-HCl, 500 mM NaCl, pH7.5). All wells were blocked with 100 μl of 3% BSA in TBS for ½ hour at room temperature. Excess BSA was removed with three TBS washes. Gp120 (purified or in supernatant) were diluted in buffer C (50 mM Tris-HCl, pH 7.5, 100 mM NaCl, 1% BSA) and added to D7324 coated plate(s) at 100μl/well. After incubating at 37° C. for 2 hours, plate(s) was washes three times with Buffer (20 mM Tris-HCl, 500 mM NaCl, 0.05% Tween-20, pH7.5) and the bound gp120 was detected by Anti-gp120-peroxidase conjugate (Cross-reactive, ImmunoDiagnostics, Inc) at a final dilution of 1:1000 in BufferC. Bound antibody-peroxidase conjugates were detected with TMB solution (Pierce) and the optical density was measured at 450 nm. Known concentration of the purified gp120IIIB (ABI) were used to generate a standard curve for gp120 quantitation.

Gp120/CD4 Binding Assay (Method for Table 1 Results)

The assay is similar to the gp120 capturing ELISA method described above with minor modifications.(43). After coating gp120 onto the plates, sCD4 was added to the plate at a final concentration of 200 ng/ml for one hour at room temperature. To determine the IC₅₀ values for entry inhibitor, the compound dissolved in DMSO was added simultaneously with sCD4. After washing with bufferB for three times, 1:1000 dilute of OKT4 (0.36 mg/ml) in Buffer C was added to the plate and the reaction was carried out for another hour at room temperature. Unbound OKT4 antibody was removed with washing buffer and anti-mouse -perxidase conjugate was added for another hour. After washing with BufferB, 100 μl of TMB solution was added and the optical density was read at 450 nm.

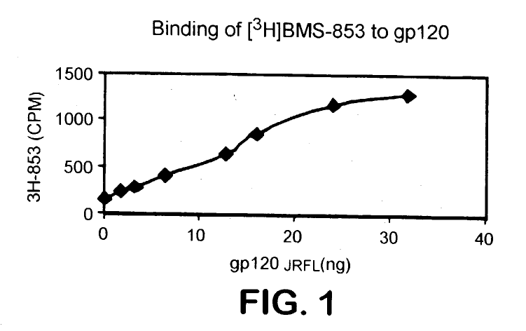

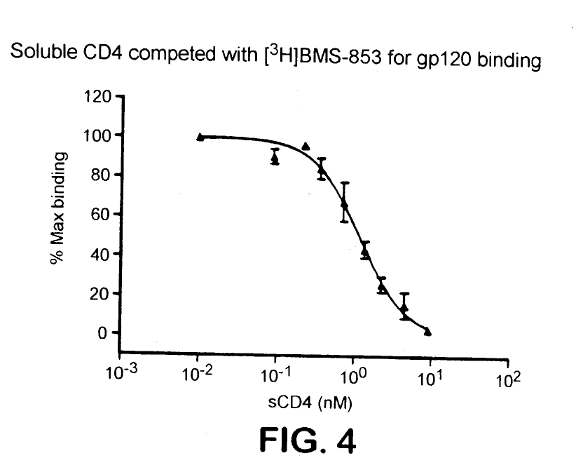

Binding of Radiolabelled [³H]BMS-853 and analogs to gp120 (method for FIGS. 1 and 4 and Table 1 Results)

FlashPlate assay. The binding of [³H]BMS-853 to gp120 was performed on FlashPlate (NEN,SMP200). Coating of gp120 onto FlashPlate was essentially the same as described for gp120 capturing ELISA. After brief washes of gp120 coated FlashPlate with TBS, [³H]BMS-853 was added to a final concentration of 16.5 nM in BufferC and the plate was kept at room temperature overnight. The plate was washed once with TBS and then counted in TopCount® Microplate Scintillation counter. Varying concentrations of gp120 were used to show the gp120 dose-dependent binding of [³H]BMS-853 (FIG. 1). Varying concentrations of sCD4 were used to demonstrate the competition between CD4 and the compound (FIG. 4).

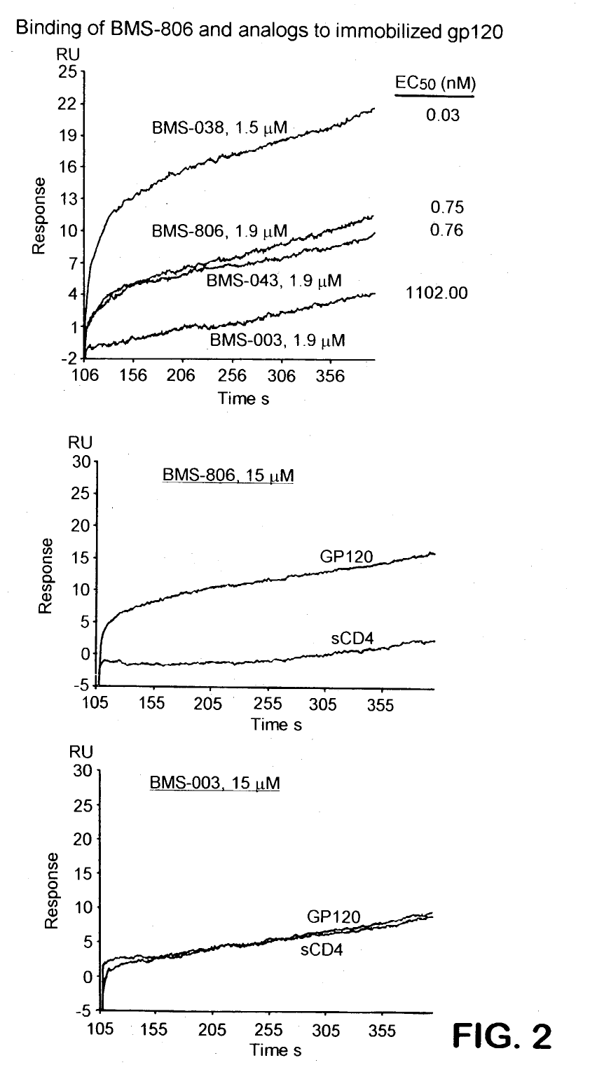

Biacore biosensor evaluation of gp120-inhibitor binding. (See FIG. 2 for results). The selective binding of entry inhibitors with gp120 was evaluated on a Biacore 3000 biosensor. Purified gp120_{JRFL} was immobilized onto the flow cell surface of a CM-5 chip via EDC/NHS activated covalent modification, according to the protocol described by Biacore Inc. (Uppsala, Swiden). The surface density of immobilized gp120 reached 12000-15000 RU. Soluble CD4 and BSA coated surfaces were also immobilized on separate flow cell surfaces as controls.

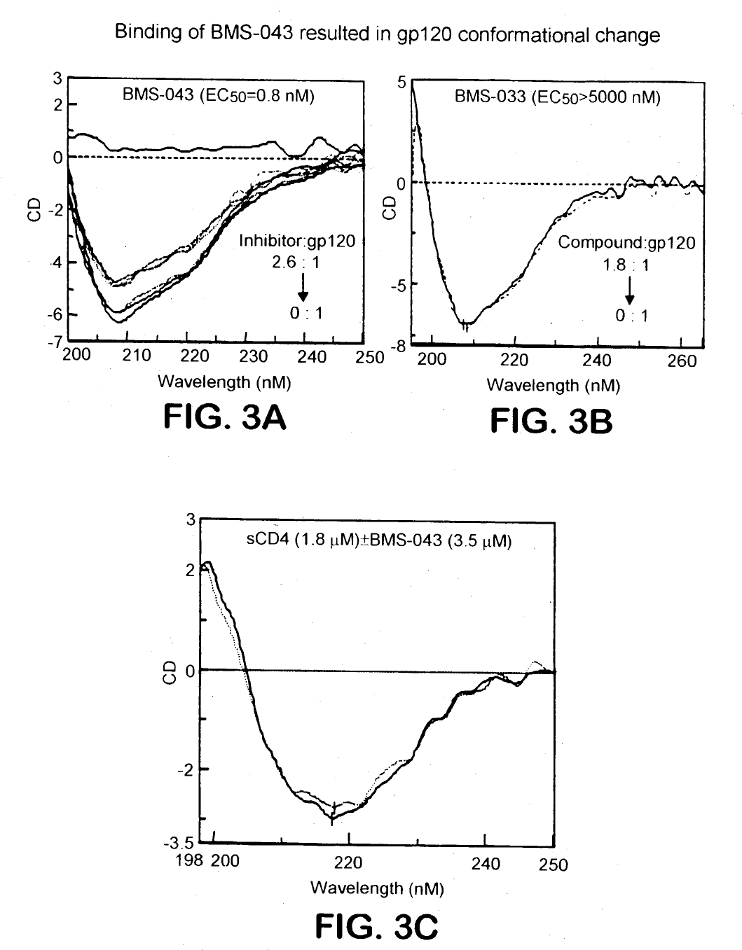

Circular Dichroism (CD) analysis. (Method for FIG. 3 results). The CD spectra of purified gp120_{JRFL} (1.2 μM in 5 mM Tris, pH 8.0/150 mM NaCl) were obtained, in the presence and absence of an inhibitor, on a Jasco J-720 spectropolarimeter at ambient (21-23° C.) temperature. Samples containing varying protein-to-compound mole ratios were placed in a quartz cuvette with a 1-mm pathlength. The molar ellipticity (θ) was monitored between 200 nm and 250 nm as the average of 12-20 scans. Soluble CD4 was used as a negative protein controls. Compound, BMS-066, with no anti-HIV activity (EC₅₀>5 μM) was included as a compound control.

Anti-HIV Single Cycle Infection Assay (Method for Table 1 Results)

Single-round infectious reporter virus was produced by co-transfecting human embryonic Kidney 293 cells with an HIV-1 envelope DNA expression vector and a proviral cDNA containing an envelope deletion mutation and the luciferase reporter gene inserted in place of HIV-1 nef sequences (Chen, 1994). Transfections were performed using LipofectAMINE PLUS reagent as described by the manufacturer (Life Technologies, Gaithersburg, Md.). The resulting reporter viruses are then used to infect HeLa/CD4/CCR5 cells in the following manner. Serial diluted compound was added to HeLa/CD4/CCR5 cells plated in 96 well plates at a cell density of 5×10⁴ cells per well in 100 μl Dulbecco's Modified Eagle Medium containing 10% fetal Bovine serum. One hundred microliter of single-round infectious reporter virus in Dulbecco's Modified Eagle Medium was then added to the plated cells and compound at a multiplicity of infection (MOI) of 0.01. Samples were harvested 72 hours after infection and viral infection was monitored by measuring luciferase expression from viral DNA in the infected cells using a luciferase reporter gene assay kit as described by the manufacturer.

Viruses and cells: (Method for Table 2 results). T-tropic (X4) strains of laboratory adapted HIV-1 were amplified in MT-2 cells and titered using a virus yield assay (17). The laboratory adapted M-tropic (R5) viruses were grown in macrophages or PM1 cells and p24 ELISA assay was used to detect the virus. The TCID₅₀/ml (tissue culture infectious dose) was calculated by the method of Spearman-Karber. Titration of viral stocks and assaying for compound sensitivity were carried out in the cell lines the viruses were originally amplified in. The 50% inhibitory concentration (EC₅₀) was calculated by using the exponential form of the median effect equation where (Fa)=1/[1+(ED₅₀/drug conc.)^{m}] (17).

Results:

Biochemical Mode of Action

Inhibition of gp120/CD4 Interaction by Entry Inhibitors (See Table 1)

Interference of bp120/sCD4 binding in the ELISA assay. To ascertain the anti-HIV entry activity of BMS-806 and analogs was the result of its interference on the gp120/CD4 interaction, we measured the binding of CD4 with gp120_{JRFL} in the presence and absence of an inhibitor using an ELISA-based binding assay. The results showed that BMS-806 inhibited the binding of sCD4 with the recombinant envelope gp120_{JRFL} with an IC50 value of 0.28μM. A parallel trend was observed between the inhibitory activity in the ELISA assay and the anti-HIV activity among various inhibitor analogs (Table 1). The results indicated that BMS-806 and analogs inhibited HIV-1 entry via interference of gp120/CD4 binding.

Binding of Entry Inhibitors to gp120. (See FIG. 1)

Binding of ³H-BMS-853 bind to gp120. To demonstrate the direct binding of an entry inhibitor to gp120, [³H]BMS-853, a closed analog of BMS-806 was synthesized and the binding activity of [³H]BMS-853 to gp120 absorbed onto the wells of a FlashPlate was investigated. As shown in FIG. 1, the level of radioactivity detected increased along with the increased concentration of gp120_{JRFL} present in the solution used to coat the plate. The results strongly indicated the direct binding of these inhibitors to both gp120_{JRFL} and gp120_{Bru}. Binding of inhibitor to covalently immobilized gp120. (See FIG. 2) The binding of inhibitors to gp120 was also evaluated using a Biacore 3000 biosensor. Purified gp120_{JRFL} was immobilized onto the flow cell surface of a CM-5 chip at a surface density of 12000-15000 RU. When solutions containing various concentrations of BMS-038 (EC₅₀≦30 pM) were flown over the pg120-coated surfaces, a dose-dependent increase of reflective index was observed. At 1.5 μM, BMS-038, the most potent among the inhibitor examined, generated the strongest signal while BMS-806, BMS-043 (EC₅₀=1 and 0.8 nM respectively) displayed intermediate signals and the weakest inhibitor BMS-038 (EC₅₀=1.1 μM) exhibited a signal level similar to that of buffer alone (FIG. 2). The flow cell surfaces were also immobilized with sCD4 as negative surface controls. When compound solutions were flown over the control surfaces the signals produced was similar to that of the buffer alone.

Conformational Change of gp120 Mediated by Inhibitor Binding. (See FIG. 3)

Comparison of the circular dichroism (CD) spectra of gp¹²⁰_{JRFL} (1.2 μM) in the absence and presence of BMS-043 (2.1 μM) showed a 40% attenuation of the protein signal mediated by the presence of the inhibitor. The inhibitor is achiral, thus optically inactive, and by itself contributed minimally to the attenuation of the protein signal. GP120 concentration was determined with Braford Protein Reagent from BioRad using BSA as the standard. A concentration-dependent increase of signal attenuation was observed as the protein/inhibitor ratio increased from 1:0 to 1:1. At protein:inhibitor ratios of 1:1, 1:2 and 1:3 the spectra overlapped with each other indicating the saturation of the inhibitor binding to gp120 (FIG. 3A). When the CD spectra of sCD4 (1.8 μM) was obtained in the presence of 3.5 μM BMS-043 no attenuation of the protein signal was observed comparing to the signal of protein alone (FIG. 3B). BMS-033 (EC₅₀>5 μM), an inactive analog of BMS-043, at 2 μM did not cause any attenuation of the gp120_{JRFL} (FIG. 3C). This selective signal attenuation mediated by the binding of BMS-043 to gp120_{JRFL} suggested the specific binding of the inhibitor resulted in a conformational change that was unique to the active entry inhibitor binding to its target protein.

Soluble CD4 Competed with BMS-806 to gp120 Binding (See FIG. 4)

Soluble CD4 was examined for its ability to interfere with ³H-BMS-853 binding to gp120. Soluble CD4 binds effectively to gp120 and the presence of BMS-853 and BMS-806 interfered with gp120/CD4 binding. It was expected that sCD4 would compete with ³H-BMS-853 for gp120 binding. As shown in FIG. 5, sCD4 at 1 μM completely inhibited H³-BMS-853 binding to gp120, with an IC50 of 1.2 nM. The fact that sCD4 inhibited H³-BMS-853/gp120 binding and BMS-853 also inhibited CD4/gp120 binding further supported that BMS-853 bind to gp120 and therefore inhibit gp120/CD4 binding.

Anti-HIV Activity

Activity of BMS-806 against HIV-1 laboratory isolates: The ability of BMS-806 to block infection of 13 different laboratory strains of B subtype HIV-1 was examined. Most of these strains used CXCR4 as a coreceptor and were analyzed in the T-cell line, MT-2. BMS-806 is very potent against 11 of the 13 laboratory strains of HIV-1 with EC₅₀ values ranging from 0.85 to 75.8 nM (Table 2). Results showed that the compound is effective against M- and T-tropic HIV-1 strains. Only two viruses, MN and RF, were not efficiently blocked by the entry inhibitor.

Chemistry

The small molecule compounds referred to herein for illustrative purposes were prepared as described below. Typical small molecule compounds having anti-HIV activity that can be used with the invention herein are disclosed in U.S. Pat. Nos. 6,469,006 and 6,476,034, both of which are incorporated by reference in their entirety.

BMS-216 is example 1 in Table 1 (activity) and Table 5 (chemistry data) in U.S. Pat. No. 6,469,006. The general procedure used to form this molecule begins at column 42 of the U.S. Pat. No. 6,469,006 patent and is excerpted below.

BMS-853 is example 39 in in Table 1 (activity) and Table 5 (chemistry data) in U.S. Pat. No. 6,469,006. The general procedure is described at the beginning of column 43 of the U.S. Pat. No. 6,469,006 patent and is appended below.

BMS-806 is described in U.S. Pat. No. 6,476,034 as compound 17a (see column 75, line 15). The synthesis proceeds through 1a, 2a, 3a, 5a, 8a, 15a, 16a and finally gives 17a. The last step of the synthesis is described on page 34 herein and all the chemistry from the application is appended below following the teachings of the U.S. Pat. No. 6,476,034 patent. Preceding steps are also appended.

BMS-216 Preparation:

### GENERAL PROCEDURE FOR PREPARATION OF EXAMPLES 1-17

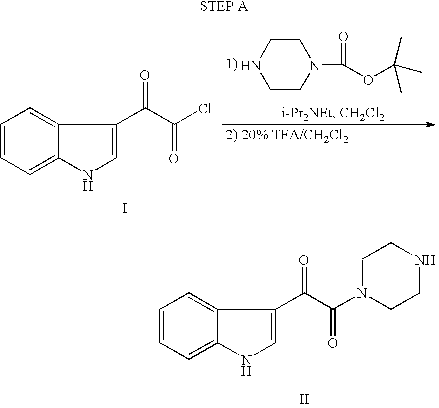

To commercially available indole-3-glyoxylyl chloride 1 (3 gram, 14.45 mmol) in CH₂Cl₂ at room temperature was added tert-butyl 1-piperazinecarboxylate (2.7 gram, 14.45 mmol) and diisopropylethylamine (2.76 ml, 15.9 mmol). The light-brown color solution was stirred for 2 hr at room temperature after which time LC/MS analysis indicated the completion of the reaction. The solvent was removed in vacuo and the resulting residue was diluted with ethyl acetate (250 ml) and diethylether (250 ml). The organic solution was then washed with water (100 ml×3) and brine (50 ml), dried over MgSO₄, filtered and concentrated. To the light-yellow solid was then added 30 ml of 20% trifluoroacetic acid in CH₂Cl₂. The solution was concentrated and the light-brown solid was dried in vacuo to give 3.5 g (95%) of product II. LC/MS analysis indicated this product was 100% pure and it was used for the next reaction without further purification.

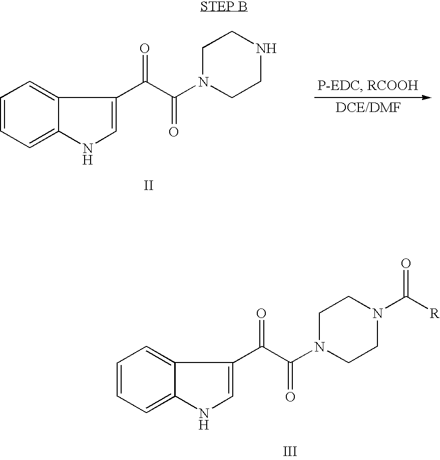

To piperazine indole-3-glyoxylamide II (0.03 mmol) was added resin-bound 1-(3-dimethylaminopropyl)-3-ethylcarbodiimide (P-EDC) (0.21 mmol) and carboxylic acid (RCOOH) (0.06 mmol) in dichloroethane (DCE) (1 mL) or DMF (dimethylformamide) (1 mL) in cases where the carboxylic acids are not soluble in DCE. The reaction was shaken for 12 hr at room temperature. The product III was filtered and concentrated. Products with purity less than 70% were diluted in methanol and purified using a Shimadzu automated preparative HPLC System. For BMS-216, R is phenyl. HPLC Retention Time is 1.13 minutes; MS data (M+H)⁺ is 362. (M+H)⁺ refers to the molecular ion peak in positive ionization mode.

BMS-853 Preparation:

### 2) GENERAL PROCEDURE FOR PREPARATION OF EXAMPLES 18-56

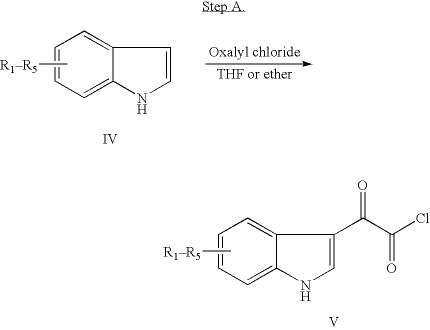

To a solution of substituted indole IV (1 eq) in dry Et₂O was dropwise added oxalylchloride (1.2 eq) at 0° C. After 5 min., the reaction mixture was warmed to room temperature, or heated to ˜35° C. overnight if necessary. The intermediate substituted-indole-3-glyoxylyl chloride V, which was formed as a solid, was filtered and washed with dry ether (2×1 ml) to remove excessive oxalyl chloride. The product was then dried under vacuum to give desired glyoxyl chlorides V.

In cases where reaction in Et₂O was unsuccessful, the following procedure was adopted: To a solution of substituted indole IV (1 eq) in dry THF (tetrahydrofuran) solvent was dropwise added oxalyl chloride (1.2 eq ) at 0° C. After 5 min., the reaction was warmed to room temperature, or heated to 70° C. under nitrogen if necessary. After concentration in vacuo, the resulting crude intermediate V was submitted to next step without further treatment.

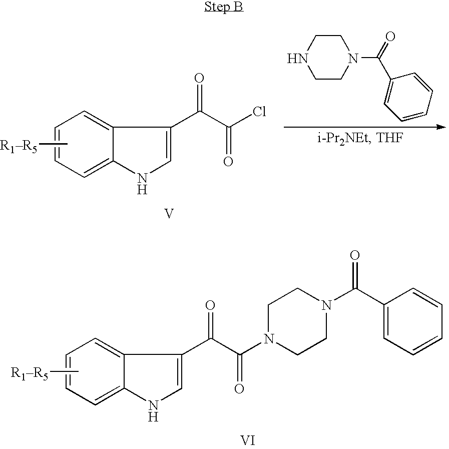

To a solution of indole glyoxyl chloride V (1 eq) in dry THF was added benzoylpiperazine (1 eq) at room temperature. Then the mixture was cooled down to 0° C., followed by dropwise addition of diisopropylamine (1.3 eq). After 5 min., the reaction mixture was warmed to room temperature and was shaken for 3 hr. The resulting crude products VI were purified by preparative HPLC and characterized. For BMS-853, R₁ and R₂ are 4.7-dimethoxy; R₃-R₅ are each hydrogen. HPLC retention time is 1.30 minutes; (M+H)⁺ is 422.

General Procedure for Preparation Additional Compounds is Provided Below:

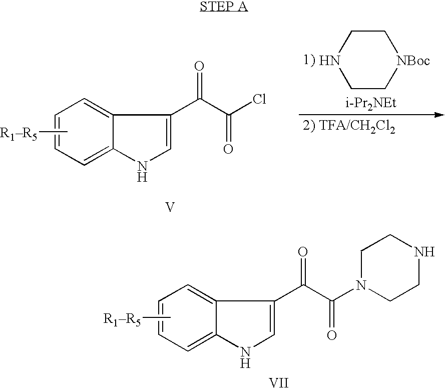

To glyoxyl chloride V (1 equiv.) in CH₂Cl₂ at room temperature was added tert-butyl 1-piperazinecarboxylate (1 equiv) and diisopropylethylamine (1.2 equiv). The solution was stirred for 2 hr at room temperature after which time LC/MS analysis indicated the completion of the reaction. The solvent was removed in vacuo and the resulting residue was diluted with ethyl acetate and diethylether. The organic solution was then washed with water (100 ml×3) and brine (50 ml), dried over MgSO₄, filtered and concentrated. To the solid was then added 30 ml of 20% trifluoroacetic acid in CH₂Cl₂. The solution was concentrated and the light-brown solid was dried in vacuo to give glyoxamide VII.

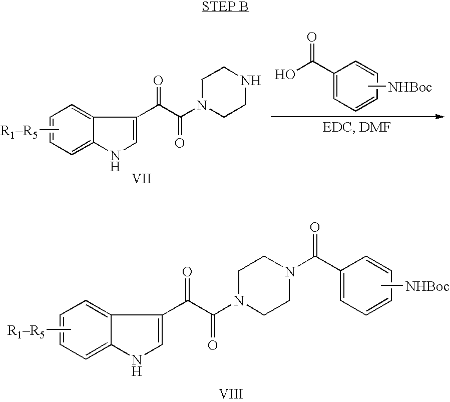

To piperazine glyoxamide VII (0.1 mmol, 1 eq) in DMF (1 mL) at room temperature was added EDC (1.5 eq) and Boc-aminobenzoic acid (1.5 eq). The reaction mixture was stirred at room temperature for 16 hours. The crude product was then purified by preparative HPLC to afford product VIII.

Preparation: BMS-806

Compound 1a is Commercially Available.

### 1) Preparation of azaindole 3-glyoxylmethyl ester 2

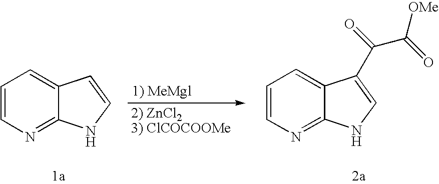

Acylation of azaindole, method A: Preparation of Methyl (7-azaindol-3-yl)-oxoacetate 2a: To a solution of 7-azaindole 1a (20.0 g, 0.169 mol) in dry CH₂Cl₂ (1000 ml), 62.1 ml of MeMgI (3.0M in Et₂O, 0.186 mol) was added at room temperature. The resulting mixture was stirred at room temperature for 1 hour before ZnCl₂ (27.7 g, 0.203 mol) was added. One hour later, methyl chlorooxoacetate (24.9 g, 0.203 mol) was injected into the solution dropwise. Then the reaction was stirred for 8 hours before being quenched with methanol.

After all solvents were evaporated, the residue was partitioned between ethyl acetate (500 ml) and H₂O (300 ml). The aqueous phase was neutralized with saturated Na₂CO₃ to pH 6-6.5, and extracted with EtOAc (3×500 ml). The organic layers were then combined, washed with 0.1N HCl (3×200 ml), dried over anhydrous MgSO₄ and concentrated in vacuo to give a crude product 2a (14.3 g, 41.5%), which was pure enough for the further reactions.

Characterization of Compounds 2:

Compound 2a, Methyl (7-azaindol-3-yl)-oxoacetate: ¹H NMR (300 MHz, DMSO-d₆) δ 8.60 (s, 1H), 8.47 (d, 1H, J=7.86 Hz), 8.40 (d, 1H, J=4.71 Hz), 7.34 (dd, 1H, J=7.86, 4.77 Hz), 3.99 (s, 3H); ¹³C NMR (75 MHz, DMSO-d₆) δ 178.7, 163.3, 149.0, 145.1, 138.8, 129.7, 119.0, 118.0, 111.2, 52.7. MS m/z: (M+H)⁺ calcd for C₁₀H₉N₂O₃: 205.06; found 205.04. HPLC retention time: 0.94 minutes (column A).

### 2) Preparation of potassium azaindole 3-glyoxylate 3

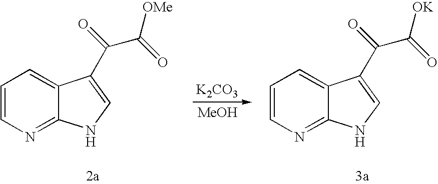

Preparation of Potassium (7-azaindol-3-yl)-oxoacetate 3a: Compound 2a (43 g, 0.21 mol) and K₂CO₃ (56.9 g, 0.41 mol) were dissolved in MeOH (200 ml) and H₂O (200 ml). After 8 hours, product 3a precipitated out from the solution. Filtration afforded 43 g of compound 3a as a white solid in 90.4% yield.

Characterization of compounds 3:

Compound 3a, Potassium (7-azaindol-3-yl)-oxoacetate: ¹H NMR (300 MHz, DMSO-d₆) δ 8.42 (d, 1H, J=7.86 Hz), 8.26 (d, 1H, J=4.71 Hz), 8.14 (s, 1H), 7.18 (dd, 1H, J=7.86, 4.71 Hz); ¹³C NMR (75 MHz, DMSO-d₆) δ 169.4, 148.9, 143.6, 135.1, 129.3, 118.2, 117.5, 112.9. MS m/z: (M+H)⁺ of the corresponding acid of compound 3a (3a−K+H) calcd for C₉H₇N₂O₃: 191.05; found 190.97. HPLC retention time: 0.48 minutes (column A).

Typical Procedure for the Preparation of Compounds in Scheme 3

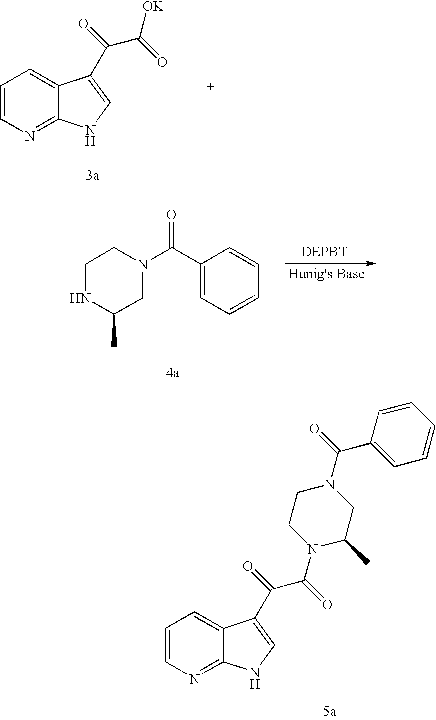

Preparation of (R)-N-(benzoyl)-3-methyl-N′-[(7-azaindol-3-yl)-oxoacetyl]-piperazine 5a: Potassium 7-azaindole 3-glyoxylate 3a (25.4 g, 0.111 mol), (R)-3-methyl-N-benzoylpiperazine 4a (22.7 g, 0.111 mol), 3-(diethoxyphosphoryloxy)-1,2,3-benzotriazin-4(3H)-one (DEPBT) (33.3 g, 0.111 mol) and Hunig's Base (28.6 g, 0.222 mol) were combined in 500 ml of DMF. The mixture was stirred at room temperature for 8 hours.

DMF was removed via evaporation at reduced pressure and the residue was partitioned between ethyl acetate (2000 ml) and 5% Na₂CO₃ aqueous solution (2×400 ml). The aqueous layer was extracted with ethyl acetate (3×300 ml). The organic phase combined and dried over anhydrous MgSO₄. Concentration in vacuo provided a crude product, which was purified by silica gel column chromatography with EtOAc/MeOH (50:1) to give 33 g of product 5a in 81% yield.

Characterization of compounds 5 with the following sub-structure:

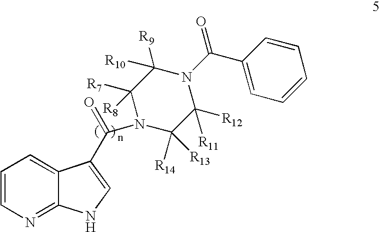

Compound 5a, n=2, R₇₋₁₃=H, R₁₄=(R)—Me, (R)—N-(benzoyl)-3-methyl-N′-[(7-azaindol-3-yl)-oxoacetyl]-piperazine: ¹H NMR (300 MHz, CD₃OD) δ □8.57 (d, 1H, J=5.97 Hz), 8.38 (d, 1H, J=4.20 Hz), 8.27 (m, 1H), 7.47 (s, 5H), 7.35 (t, 1H, J=5.13 Hz), 4.75-2.87 (m, 7H), 1.31 (b, 3H); ¹³C NMR (75 MHz, CD₃OD) δ 185.6, 172.0, 166.3, 148.9, 144.6, 137.0, 134.8, 130.2, 129.9, 128.4, 126.6, 118.6, 118.0, 112.2, 61.3, 50.3, 45.1, 35.5, 14.9, 13.7. MS m/z: (M+H)⁺ calcd for C₂₁H₂₁N₄O₃: 377.16; found 377.18. HPLC retention time: 1.21 minutes (column A).

### 1) N-Oxide formation (equation 1, Scheme 5)

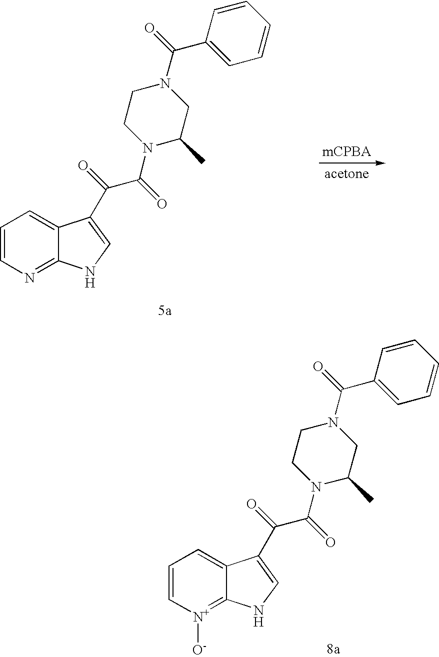

Preparation of (R)—N-(benzoyl)-3-methyl-N′-[(7-oxide-7-azaindol-3-yl)-oxoacetyl]-piperazine 8a: 10 g of 7-azaindole piperazine diamide 5a (26.6 mmol) was dissolved in 250 ml acetone. 9.17 g of mCPBA (53.1 mmol) was then added into the solution. Product 8a precipitated out from the solution as a white solid after 8 hours and was collected via filtration. After drying under vacuum, 9.5 g of compound 8a was obtained in 91% yield. No further purification was needed.

Characterization of compound 8 with he following sub-structure:

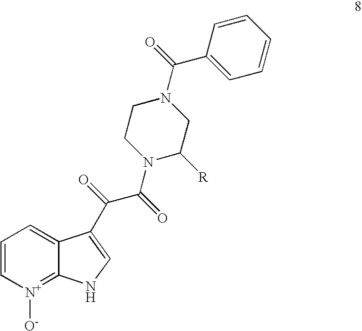

Compound 8a, R═(R)—Me, (R)—N-(benzoyl)-3-methyl-N′-[(7-oxide-7-azaindol-3-yl)-oxoacetyl]-piperazine: ¹H NMR (300 MHz, DMSO-d₆) δ 8.30 (d, 1H, J=12.2 Hz), 8.26 (d, 1H, J=10.1 Hz), 8.00 (d, 1H, J=7.41 Hz), 7.41 (s, 5H), 7.29 (m, 1H), 4.57-2.80 (m, 7H), 1.19 (b, 3H); ¹³C NMR (75 MHz, DMSO-d₆) δ 186.2, 170.0, 165.0, 139.5, 136.9, 136.7, 135.5, 133.5, 129.7, 128.5, 126.9, 121.6, 119.9, 113.6, 49.4, 44.3, 15.9, 14.8. MS m/z: (M+H)⁺ calcd for C₂₁H₂₁N₄O₄: 393.16; found 393.16. HPLC retention time: 1.05 minutes (column A).

### 3) Nitration of N-Oxide (equation 10, Scheme 6)

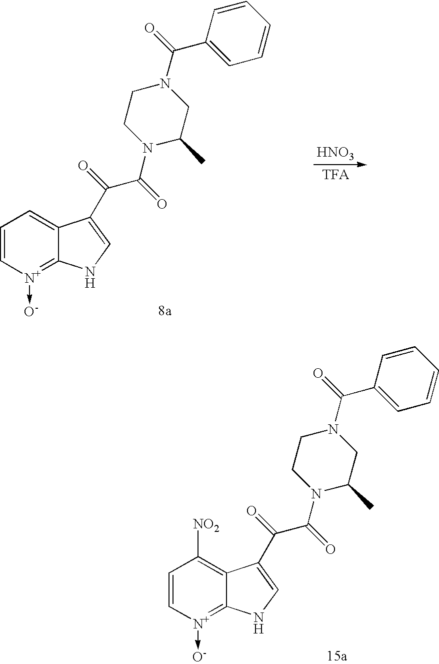

Preparation of (R)—N-(benzoyl)-3-methyl-N′-[(4-nitro-7-oxide-7-azaindol-3-yl)-oxoacetyl]-piperazine 15a: N-oxide 8a (10.8 g, 27.6 mmol) was dissolved in 200 ml of trifluoroacetic acid and 20 ml of fuming nitric acid. The reaction mixture was stirred for 8 hours and quenched with methanol. After filtration, the filtrate was concentrated under vacuum to give crude product 15a as a brown solid, which was carried to the next step without further purification. A small amount of crude product was purified using a Shimadzu automated preparative HPLC System to give compound 3 mg of compound 15a.

Characterization of compound 15 with the following sub-structure:

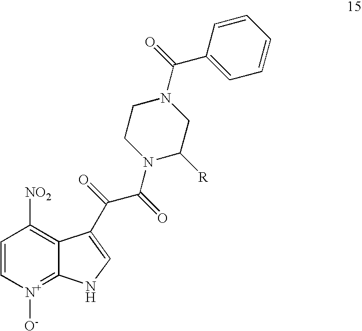

Compound 15a, R═(R)—Me, (R)—N-(benzoyl)-3-methyl-N′-[(4-nitro-7-oxide-7-azaindol-3-yl)-oxoacetyl]-piperazine: MS m/z: (M+H)⁺ calcd for C₂₁H₂₀N₅O₆: 438.14; found 438.07. HPLC retention time: 1.18 minutes (column A).

### 9) Displacement of Nitro Group (equation 11, Scheme 6)

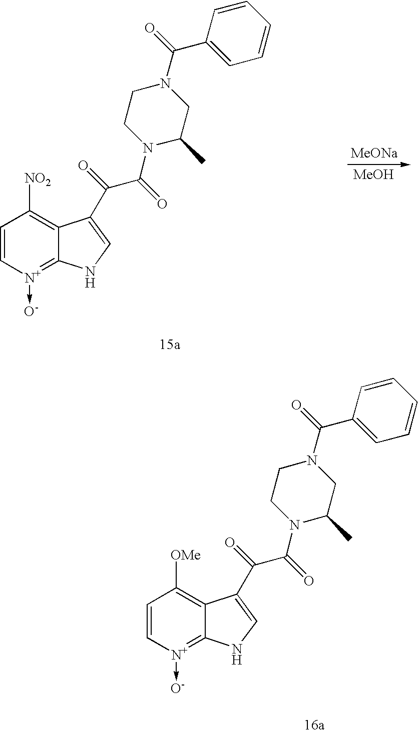

Preparation of (R)—N-(benzoyl)-3-methyl-N′-[(4-methoxy-7-oxide-7-azaindol-3-yl)-oxoacetyl]-piperazine 16a: 100 mg of crude compound 15a from the previous step was dissolved in 6 ml of 0.5M MeONa in MeOH. The reaction mixture was refluxed for 8 hours, and the solvent removed under vacuum to afford a mixture including product 16a and other inorganic salts. This mixture was used in the next step without further purification. A small portion of the crude mixture was purified using a Shimadzu automated preparative HPLC System to give 5 mg of compound 16a.

Characterization of compounds 16 with the following sub-structure:

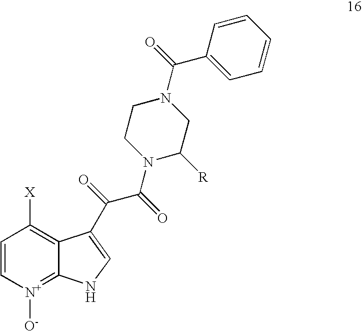

Compound 16a, X═OMe, R═(R)—Me, (R)—N-(benzoyl)-3-methyl-N′-[(4-methoxy-7-oxide-7-azaindol-3-yl)-oxoacetyl]-piperazine: MS m/z: (M+H)⁺ calcd for C₂₂H₂₃N₄O₅ 423.17, found 423.04. HPLC retention time: 0.97 minutes (column A).

### 10) Reduction of N-Oxide (equation 12, Scheme 6)

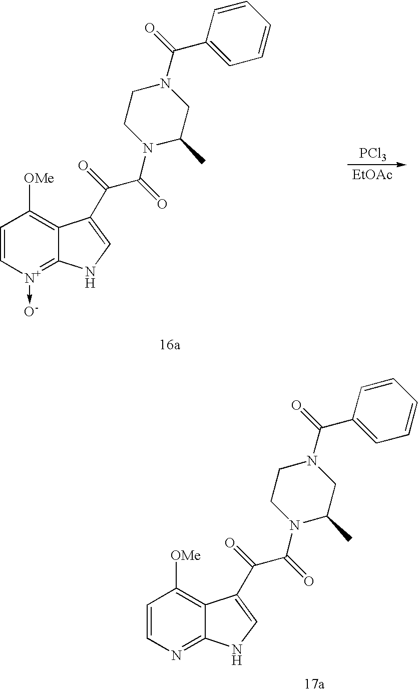

Preparation of (R)—N-(benzoyl)-3-methyl-N′-[(4-methoxy-7-azaindol-3-yl)-oxoacetyl]-piperazine 17a: 48 mg of crude 16a was suspended in 30 ml of ethyl acetate at room temperature. 1 ml of PCl₃ was added and the reaction was mixture stirred for 8 hours. The reaction mixture was poured into ice cooled 2N NaOH solution with caution. After separating the organic layer, the aqueous phase was extracted with EtOAc (6×80 ml). The organic layers were combined, and concentrated in vacuo to give a residue which was purified using a Shimadzu automated preparative HPLC System to give 38 mg of compound 17a.

Characterization of compounds 17 with the following sub-structure:

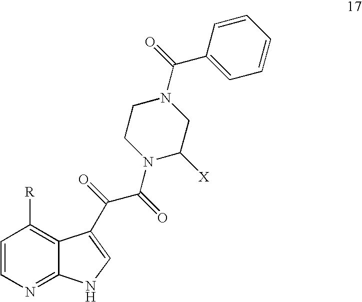

Compound 17a, R═Ome, X═(R)—Me, (R)—N-(benzoyl)-3-methyl-N′-[(4-methoxy-7-azaindol-3-yl)-oxoacetyl]-piperazine: ¹H NMR (300 MHz, CD₃OD) δ 8.24 (d, 1H, J=5.7 Hz), 8.21(m, 1H), 7.47 (s, 5H), 6.90 (d, 1H, J=5.7 Hz), 4.71-3.13 (m, 10H), 1.26 (b, 3H); ¹³C NMR (75 MHz, CD₃OD) δ 185.3, 172.0, 167.2, 161.2, 150.7, 146.6, 135.5, 134.8, 129.9, 128.3, 126.7, 112.8, 106.9, 100.6, 54.9, 50.2, 48.1, 45.1, 14.5, 13.8. MS m/z: (M+H)⁺ calcd for C₂₂H₂₃N₄O₄: 407.17; found 407.19. HPLC retention time: 1.00 minutes (column A).

The other compounds can also be made according to the procedures above in a similar manner. The compound structures are provided below:

| | | |
|---|---|---|
| | | formula |
| BMS Number | Structure | weight |

| | | | |
|---|---|---|---|
| BMS-806 | 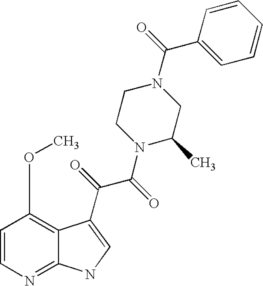 | Chiral | 406.45 |

| | | |
|---|---|---|
| BMS-216 | 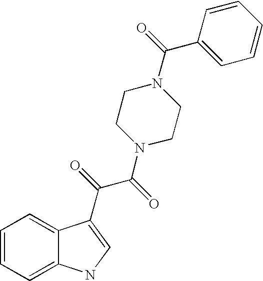 | 361.4 |
| BMS-043 | 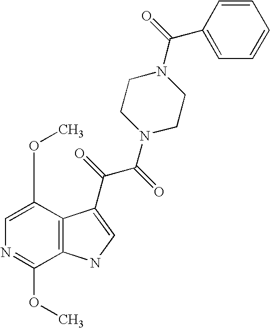 | 422.44 |
| BMS-033 | 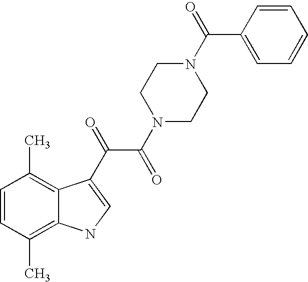 | 389.46 |
| BMS-003 | 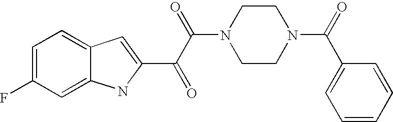 | 379.39 |
| BMS-038 | 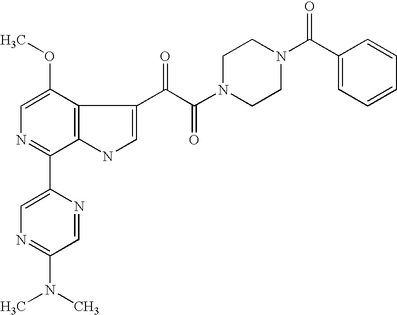 | 513.56 |
| BMS-853 | 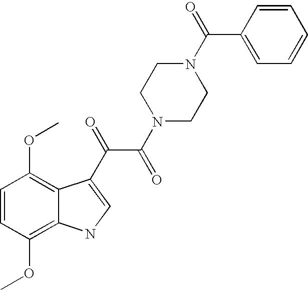 | 421.46 |

What is claimed is:

1. A method of inhibiting HIV infection in a mammal by administering to said mammal in need thereof a small molecule compound having a molecular weight of less than about 1,000 dalton, wherein said compound interacts with HIV-gp120 in such a manner as to cause conformational change in said gp120 thereby preventing interaction between said gp¹²⁰ and leukocyte CD4.

2. The method of claim 1 wherein said compound has a molecular weight of less than about 750 dalton.

3. The method of claim 2 wherein said compound has a molecular weight of less than about 500 dalton.

4. The method of claim 1 wherein said compound is administered orally to said mammal.

5. The method of claim 1 wherein said compound is selected from the group consisting of compounds BMS-806, BMS-216, BMS-043, BMS-033, BMS-003, BMS-038 and BMS-853.
